(19) United States
(12) Patent Application Publication
(10) Pub. No.: US 2025/0017766 A1
(43) Pub. Date: Jan. 16, 2025

(54) PATIENT TARGETED TEMPERATURE MANAGEMENT DEVICE AND METHOD

(71) Applicant: Vikram Singh Bhinder, Greenville, NC (US)

(72) Inventor: Vikram Singh Bhinder, Greenville, NC (US)

(21) Appl. No.: 18/904,647

(22) Filed: Oct. 2, 2024

Related U.S. Application Data
(63) Continuation-in-part of application No. 18/640,583, filed on Apr. 19, 2024.
Application No. 18/640,583 is a continuation-in-part of application No. 17/370,143, filed on Jul. 8, 2021, now abandoned.

(51) Int. Cl. A61F 7/00

(52) U.S. Cl. CPC A61F 7/0085; A61F 2007/0008; A61F 2007/0011; A61F 2007/0056

(57) ABSTRACT

A medical device and method used for patient temperature management, wherein the device is designed to cool the patient's head and blood flow to the head and neck including the two carotid and the two vertebral arteries of a patient after trauma in order to prevent inflammation and reduce swelling in the brain. It is portable with a rapid cooling capability feature, does not require an ICU setting, eliminates certain complications that could affect the patient otherwise, and can be quickly and easily placed on the patient with little training or expertise.

This application is a continuation-in-part of U.S. non-provisional application Ser. No. 18/640,583 filed on Apr. 19, 2024, which is a CIP of U.S. application Ser. No. 17/370,143 filed on Jul. 8, 2021. This application also claims priority of PCT/US2022/34496 filed on Jun. 22, 2022, which claims priority of U.S. non-provisional application Ser. No. 17/370,143 filed on Jul. 8, 2021, all of which is incorporated herein in their entirety by reference.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

### Field of the Invention

The present invention relates to a device and method for patient temperature management. In particular, it is a medical device designed to cool blood flow to the head and neck area, including the two carotid arteries and the two vertebral arteries during/after a trauma, cardiac arrest, stroke, heart surgery, seizures, other diseases, and the like, to prevent and treat brain injury.

### Description of Related Art

Therapeutic hypothermia is well known to have neuroprotective effects in several situations including cerebral ischemia, cardiac arrest, and neonatal asphyxia. Therapeutic hypothermia buys the patient more time and can make a huge difference in outcomes. During the life-saving process, a patient's body temperature is cooled to about 93º Fahrenheit and kept at that temperature for around 24 hours; an approach that decreases both inflammation and reduces brain swelling.

The basic mechanisms through which therapeutic hypothermia protects the brain and other tissues are clearly multi-factorial and include at least the following: reduction in metabolic rate, effects on blood flow, reduction of the critical threshold for oxygen delivery, blockade of excitotoxic mechanisms, calcium antagonism, preservation of protein synthesis, reduction of brain thermopooling, a decrease in edema formation, modulation of the inflammatory response, neuroprotection of the white matter, and modulation of apoptotic cell death.

This complex, time-intensive therapy requires specialized training and equipment, and typically is only provided in a clinical setting since the equipment involved is neither portable nor easy to use. Current devices for patient-targeted temperature management have several drawbacks namely:
a) placement can take more than 30 minutes;
b) portable capability is not an option;
c) an ICU (intensive care unit) setting is required;
d) achieving target temperature can take hours;
e) there is an association with complications such as pneumonia due to long treatment times and whole body cooling;
f) there is a restriction of use in patients with blood coagulation issues;
g) they do not cover the two carotid arteries and the two vertebral arteries; and
h) none of the current devices cool both the two carotid arteries, the two vertebral arteries, the neck, and the head simultaneously.

### BRIEF SUMMARY OF THE INVENTION

The present invention relates to a device and method which provides therapeutic hypothermia specifically to a patient's head and neck, the two carotid arteries and the two vertebral arteries, is portable, takes little training or expertise, mounts to the patient′ head quickly, can cool all or part of the head as desired with the exception of the face, has a rapid cooling capability compared to current methods and devices, has an easy placement feature, and can be easily placed on a patient outside of an ICU, even by emergency medical technicians. In one embodiment, the rapid cooling time is about 30 minutes to about 60 minutes.

Accordingly, in one embodiment, there is a device for providing rapid therapeutic hypothermia to a patient's head and neck comprising:
a) a covering that covers the head and neck including the two carotid arteries and the two vertebral arteries of the patient, with the exception of the face of the patient, such that it cools the brain, the two carotid arteries and the two vertebral arteries to the brain, wherein there are two or more side by side compartments in a single layer, each compartment capable of accepting a CO₂ cooled liquid that is between −30° C. and 40° C. and wherein the compartments are constructed to produce a turbulent flow in each compartment and wherein there are thickenings in the neck component along the anatomical course of carotid artery such that it presses on carotid arteries with a tension meter attached to the front of neck portion to tighten the neck portion;
b) at least one inlet tubing for each compartment designed to deliver cooled liquid to the compartments;
c) at least one outlet tubing for each compartment designed to remove the cooled liquid from the compartment and return it to the source of the CO₂ cooled liquid; and
d) a source of CO₂ cooled liquid, which delivers the CO₂ cooled liquid to the at least one inlet tubing.

In another embodiment, there is a method for providing rapid therapeutic hypothermia to a patient's head and neck including two carotid arteries and the two vertebral arteries comprising:
a) a single pre-cooled liquid cooled by a CO₂ source;
b) delivering the cooled liquid to two or more side by side compartments in a head covering, which covers the head and neck of the patient, the two carotid arteries and the two vertebral arteries of the patient with the exception of the face of the patient, such that it cools the brain, the two carotid arteries and the two vertebral arteries to the brain and wherein there are 2 thickenings in the neck coverage such that each thickening presses on a carotid artery;
c) creating turbulence in the cooled liquid as it passes through the two or more compartments; and
d) removing the cooled liquid from the two or more compartments.

### BRIEF DESCRIPTION OF THE DRAWINGS

FIG. 1 is a side perspective of the temperature management device of the present invention.

FIG. 2 is a top view of the temperature management device of the present invention.

FIG. 3 is a see-through view of the tubing of the present invention, showing turbulent flow.

FIG. 4 is a side view of the thickening pressing on the neck near the carotid artery and vertebral artery.

FIG. 5 is a view of the human head showing the portion of the device where the thickening presses against the carotid artery.

FIG. 6 is a view of optional tubes for wetting the hair of the patient.

### DETAILED DESCRIPTION OF THE INVENTION

While this invention is susceptible to embodiment in many different forms, there is shown in the drawings, and will herein be described in detail, specific embodiments with the understanding that the present disclosure of such embodiments is to be considered as an example of the principles and not intended to limit the invention to the specific embodiments shown and described. In the description below, like reference numerals are used to describe the same, similar, or corresponding parts in the several views of the drawings. This detailed description defines the meaning of the terms used herein and specifically describes embodiments in order for those skilled in the art to practice the invention.

### Definitions

The terms “about” and “essentially” mean±10 percent.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

The term “comprising” is not intended to limit inventions to only claiming the present invention with such comprising language. Any invention using the term comprising could be separated into one or more claims using “consisting” or “consisting of” claim language and is so intended.

Reference throughout this document to “one embodiment”, “certain embodiments”, “an embodiment”, or similar terms means that a particular feature, structure, or characteristic described in connection with the embodiment is included in at least one embodiment of the present invention. Thus, the appearances of such phrases in various places throughout this specification are not necessarily all referring to the same embodiment. Furthermore, the particular features, structures, or characteristics may be combined in any suitable manner in one or more embodiments without limitation.

The term “or”, as used herein, is to be interpreted as an inclusive or meaning any one or any combination. Therefore, “A, B, or C” means any of the following: “A; B; C; A and B; A and C; B and C; A, B, and C”. An exception to this definition will occur only when a combination of elements, functions, steps, or acts are in some way inherently mutually exclusive.

The drawings featured in the figures are for the purpose of illustrating certain convenient embodiments of the present invention and are not to be considered as limitation thereto. The term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein, and use of the term “means” is not intended to be limiting.

As used herein, the term “therapeutic hypothermia” refers to targeted temperature management (TTM), which is an active treatment that tries to achieve and maintain a specific cooler head/neck temperature of about −30° C. to about 40° C. as compared to normal human temperatures in a person for a specific duration of time. Temperature can be measured in any place that can give an accurate temperature reading such as in the nasopharynx, brain parenchyma using a brain temperature monitor, or the inner ear. This technique is well known, however, devices used to achieve therapeutic hypothermia are usually not portable, lack rapid cooling, nor are they easy to use. In the present invention, therapeutic hypothermia is achieved by use of a device and method that actively prevents/treats brain injury (e.g., stroke) using a coolant carbon dioxide (CO₂) and a unique capture cover to be positioned over the head and neck, the two carotid arteries and the two vertebral arteries. This is done in an effort to improve health outcomes due to cessation of blood flow to the brain, which can occur from numerous other causes including, but not limited to cardiac arrest, stroke, seizures, brain hemorrhages, and high intracranial pressures and for which therapeutic hypothermia improves survival and brain function. In published literature, therapeutic hypothermia post cardiac arrest having a core body temperature of both 33° C. (91° F.) and 36° C. (97° F.) appear to result in similar outcomes. Any lower core body temperature of less than 32° C. is associated with significant complications, including cessation of heart activity. Hence, safe lower core body temperatures of less than 32° C. are possible only when the patient is on a heart/lung machine. The present invention is a device and method that make rapid brain cooling to lower temperatures possible as the rest of the body could be counter warmed, thus achieving better results with minimal complications. Therapeutic hypothermia is thought to prevent brain injury by several methods including decreasing the brain's oxygen demand, reducing the production of neurotransmitters like glutamate, as well as reducing free radicals that might damage the brain. The present device can be placed in seconds, begins cooling the head and neck, (including the two carotid arteries and the two vertebral arteries) area in less than one minute, improves survivability, reduces the chance of brain injury, lowers pressure in the brain, can be done at home, and lowers the risk of seizures. The device can be used to treat neurological and cardiological emergencies such as:
a. Cardiac arrest;
b. Seizure;
c. Stroke;
d. Trauma;
e. Intracranial hypertension; and
f. Cardiological surgeries.

As used herein, the term “patient's head and neck” refers to the head and neck of a patient, including the carotid and vertebral arteries, in need of therapeutic hypothermia excluding the face, as shown in the figures.

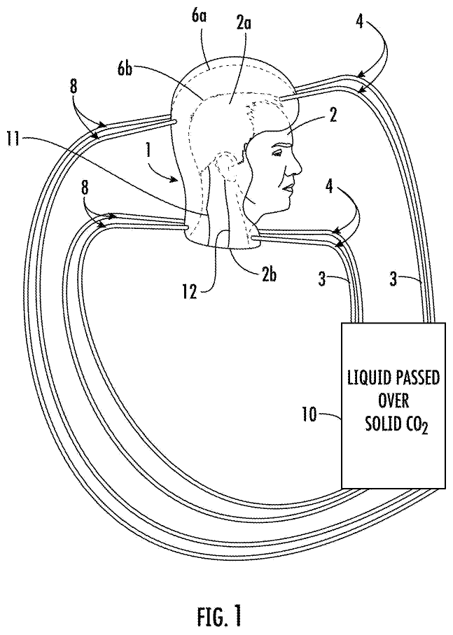

As used herein, the term “covering that covers the head and neck of the patient” refers to the design of the device, such that it covers the head and neck with the exception of the facial area of the head, but includes the two carotid arteries and the two vertebral arteries being covered as shown in FIG. 1. It also includes a thickening along the anatomical course of the arteries to improve cooling of the carotid arteries. This provides optimal pressure without occluding the arteries. It can be constructed of silicone or other medically acceptable material that is impervious to cooled liquid. The cover will have two or more side by side compartments, each of which creates turbulent flow of cooled liquid in the compartments.

As used herein, the term “two or more compartments” refers to the fact that the device can have separate independent areas for cooling, such that one or more of the compartments are utilized without cooling an area of the head and neck that is not necessary. At least one inlet tubing and one outlet tubing per compartment is necessary. In one embodiment, there are two side by side compartments as shown in the Figures. In this view, there is a left and right hemisphere compartment. However, any number of two or more is contemplated as long as they are on one level.

As used herein, the term “CO₂ cooled liquid” refers to a liquid like acetone, water, and the like passing over CO₂ which cools the liquid and the CO₂ cooled liquid passes through the tubes into the compartments (having turbulent flow), while the chilled acetone at a temperature of about −30° C. to about +40° C. cools/re-warms the head and neck, the two carotid arteries and the two vertebral arteries in contact with each compartment. The outlet tubing returns the cooled liquid to the source for re-cooling. In one embodiment, the device can be used to re-warm the patient's head and neck by utilizing a warmer liquid instead of a cold liquid. This method of cooling has not been used for medical purposes or in any other devices. The new implementation of this method and device allows the cooled liquid to flow through tubes and enter the compartments, which creates turbulence to sufficiently cool the head and neck to targeted temperatures in each compartment before exiting the compartment and returning to the source.

As used herein, the term “independent of each other” refers to each of the compartments noted above operating entirely independent of any other compartment. Thus, each compartment is fitted with one or more of the inlet tubing and outlet tubing for cooling the corresponding compartment (i.e., CO₂ cooled acetone or cooled liquid passing through at least one tube per compartment). It is also fitted with a device to produce turbulent flow of the cooled liquid in the compartment (e.g., by placing baffles in the compartment as shown in FIG. 3).

As used herein, the term “inlet tubing” refers to tubing that passes CO₂ cooled liquid into one of the compartments of the device. From there, the CO₂ liquid exits the compartment through outlet tubing and passes back to the original source of cooling liquid in order to re-cool the liquid. The tubing can be made from silicone or any other acceptable material that is essentially impervious to the CO₂ cooled acetone or other cooled liquids. In one embodiment, the tubing is insulated to help prevent premature heating of the cooled liquid.

As used herein, the term “outlet tubing” refers to tubing exiting the compartment that allows the CO₂ cooled liquid to leave the compartment to go to an acceptable location (the air, a container, or the like). In one embodiment, the cooled liquid is returned to the source for re-cooling.

As used herein, the term “turbulent flow” refers to baffles or other obstructions in the compartments that cause the CO₂ cooled liquid to swirl or agitate to achieve maximal heat exchange to improve the cooling effect on the patient.

As used herein, the term “source of CO₂ cooled liquid” refers to a source of acetone, water, and the like passed over CO₂. In addition, it has a pump or other means such as flow pressure, temperature sensors, and the like to feed the CO₂ cooled liquid to the inlet tubing linking the source to the head/neck compartment.

As used herein, the term “delivers carbon dioxide (CO₂) cooled liquid to the at least one inlet tubing” refers to the source of cooled liquid being able to pump, or by pressure, feed cooled liquid to one or more of the inlet tubing in the device to deliver the cooled liquid to the compartments.

As used herein, the term “thickening” refers to a bulge in the neck portion of the cover such that the thickening presses on the neck where each carotid artery is in order to improve the cooling of the carotid artery.

As used herein, the term “tension meter” is a device used to measure tension in wires, cables, textiles, mechanical belts and more. In one embodiment, the tension meter is handheld.

As used herein the term “tube” refers to a tube with holes positioned on the hair of the patient for wetting the hair for improved thermal conductivity. In one embodiment, there are holes in the tube for delivering water to the head of the patient.

As used herein the term “solvent” refers to water, saline or any other liquid that improves conductivity and heat transfer between the skin and the pad.

### DRAWINGS

Now referring to the drawings, FIG. 1 is a side perspective of the temperature management device of the present invention 1 mounted on the head 2a and neck 2b of patient 2 including the carotid and vertebral arteries. In this view, liquid 3 is passed over CO₂ to produce a cooled liquid 10 which is delivered to inlet tubing 4. From there, CO₂ cooled liquid travels to the side by side compartments 6a and 6b where it encounters turbulent flow to provide the desired therapeutic hypothermia to a patient's head and neck. From there, the CO₂ cooled liquid exits the compartments via the outlet tubing 8 and returns to the CO₂ to re-cool the liquid. In this embodiment, there are two inlets 4 in the top of the head 2a and two inlets in the neck 2b region of patient 2, but more are within the scope of this invention. Shown in this view is carotid artery 12 and vertebral artery 11.

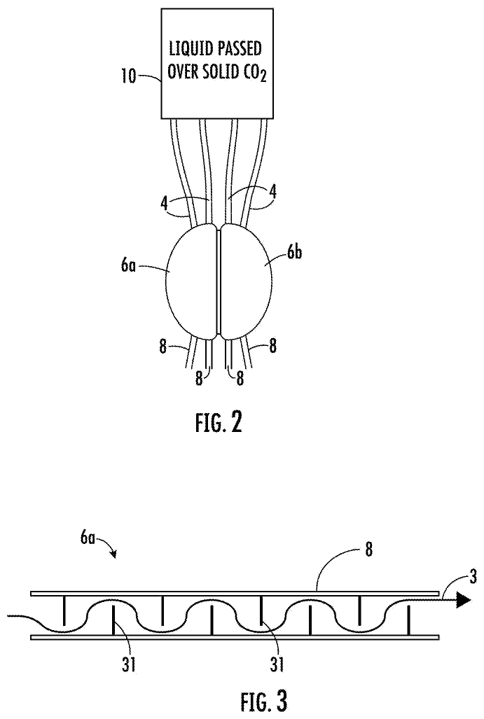

FIG. 2 is a top view of the temperature management device of the present invention 1 clearly showing compartments 6a and 6b. FIG. 3 is a see-through section of compartment 6a, which shows examples of baffles 31 in tubing 8 for creating turbulent flow of the CO₂ cooled liquid 10 traveling through the tube 8 and being agitated because of the baffles 31.

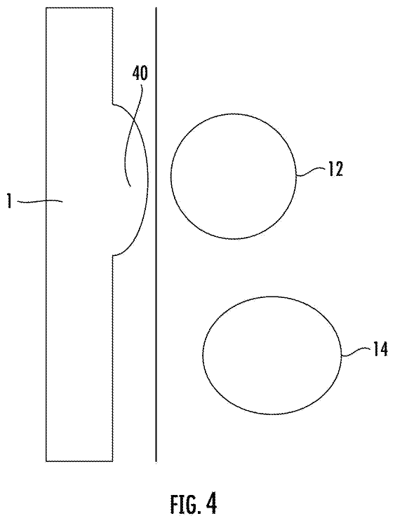

FIG. 4 is a side view wherein the thickening 40 presses on the neck 2b near the carotid artery 12 and vertebral artery 14. This improves the cooling of carotid artery 12 and vertebral artery 14.

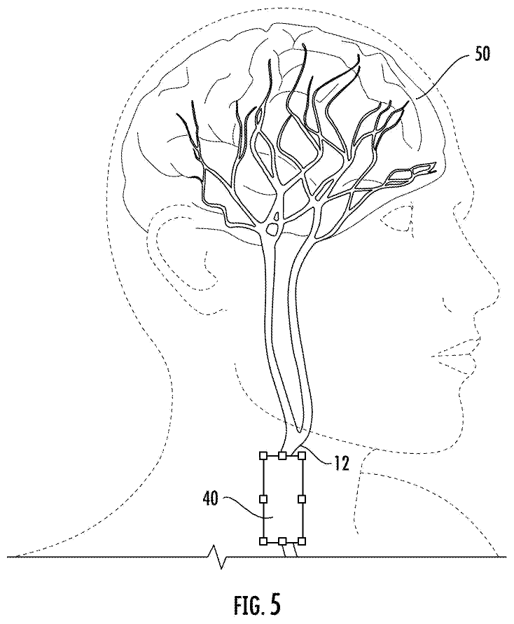

FIG. 5 is a view of the human head 50 showing the portion of the device where the thickening presses against the carotid artery.

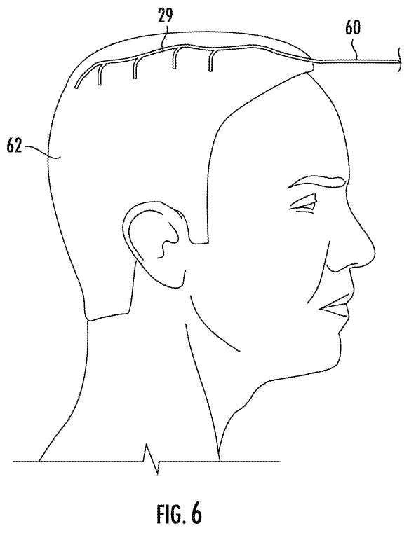

FIG. 6 is the optional tubes with holes 60 positioned on the hair 62 so that the hair of the patient can be wetted for better thermal conduction.

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials, and the like apparent to those skilled in the art still fall within the scope of the invention as claimed by the applicant.

What is claimed is:

1. A device for providing rapid therapeutic hypothermia to a patient's head and neck comprising:
a) a covering that covers the head and neck, including the two carotid arteries and the two vertebral arteries of the patient, with the exception of the face of the patient, such that it cools the brain, the two carotid arteries and the two vertebral arteries to the brain, wherein there are two or more side by side compartments in a single layer, each compartment capable of accepting a CO₂ cooled liquid that is between −30° C. and 40° C. and wherein the compartments are constructed to produce a turbulent flow in each compartment and wherein there are thickenings in the neck component along the anatomical course of carotid artery such that it presses on carotid arteries with a tension meter attached to the front of neck portion to tighten the neck portion;
b) at least one inlet tubing for each compartment designed to deliver cooled liquid to the compartments;
c) at least one outlet tubing for each compartment designed to remove the cooled liquid from the compartment and return it to the source of the CO₂ cooled liquid; and
d) a source of CO₂ cooled liquid, which delivers the CO₂ cooled liquid to the at least one inlet tubing.

2. The device for providing rapid therapeutic hypothermia to a patient's head and neck according to claim 1, wherein there is a left hemisphere and a right hemisphere compartment.

3. The device for providing rapid therapeutic hypothermia to a patient's head and neck according to claim 1, wherein there are 4 inlet tubes and 4 outlet tubes.

4. The device for providing rapid therapeutic hypothermia to a patient's head and neck according to claim 1, wherein turbulent flow is created by a plurality of baffles in the compartments.

5. The device for providing rapid therapeutic hypothermia to a patient's head and neck according to claim 1, wherein at least one of a portion of the inlet or outlet tubing is insulated.

6. The device for providing rapid therapeutic hypothermia to a patient's head and neck according to claim 1, wherein the cooled liquid is acetone.

7. The device for providing rapid therapeutic hypothermia to a patient's head and neck according to claim 1, wherein the device is used to re-warm the patient's head and neck.

8. The device for providing rapid therapeutic hypothermia to a patient's head and neck according to claim 1, wherein the device is portable.

9. A method for providing rapid therapeutic hypothermia to a patient's head and neck including two carotid arteries and the two vertebral arteries comprising:
a) a single pre-cooled liquid cooled by a CO₂ source;
b) delivering the cooled liquid to two or more compartments in a head covering, which covers the head and neck of the patient, the two carotid arteries and the two vertebral arteries with the exception of the face of the patient, such that it cools the brain, the two carotid arteries and the two vertebral arteries to the brain and wherein there are 2 thickenings in the neck coverage such that each thickening presses on a carotid artery;
c) creating turbulence in the cooled liquid as it passes through the two or more compartments; and
d) removing the cooled liquid from the two or more compartments.

10. The method according to claim 9, wherein the removed cooled liquid is returned to the CO₂ source to re-cool.

11. The method according to claim 9, wherein the cooled liquid is acetone.

12. The method according to claim 9, which is used in neurological and cardiological emergencies.

13. The method according to claim 12, wherein the emergencies are selected from the group comprising:
a) Cardiac arrest;
b) Seizure;
c) Stroke;
d) Trauma;
e) Intracranial hypertension; and
f) Cardiological surgeries.

14. The device according to claim 1 which can be used to treat cardiac and neurological emergencies.

15. The device according to claim 14, wherein the emergencies are selected from the group comprising:
a) Cardiac arrest;
b) Seizure;
c) Stroke;
d) Trauma;
e) Intracranial hypertension; and
f) Cardiological surgeries.

16. The device according to claim 1 which further comprises a tube which can be positioned on the top of the patient's head, designed to deliver water to the head.

17. The device according to claim 16 wherein the tube has a plurality of holes to deliver water or a solvent to the patient's head.
